(19) United States
(12) Patent Application Publication
(10) Pub. No.: US 2023/0309931 A1
(43) Pub. Date: Oct. 5, 2023

(54) DEEP LEARNING-BASED SIMPLE URINE FLOW TEST RESULT LEARNING METHOD AND LOWER URINARY TRACT SYMPTOM DIAGNOSIS METHOD

(71) Applicant: SAMSUNG LIFE PUBLIC WELFARE FOUNDATION, Seoul (KR)

(72) Inventors: Kyu Sung LEE, Seoul (KR); Deok Hyun HAN, Seoul (KR); Baek Hwan CHO, Seoul (KR); Seok Hwan BANG, Seoul (KR)

(21) Appl. No.: 18/029,236

(22) PCT Filed: Oct. 15, 2021
(86) PCT No.: PCT/KR2021/014355

§ 371 Date: Mar. 29, 2023

(30) Foreign Application Priority Data
Oct. 23, 2020 (KR) 10-2020-0138647

(51) Int. Cl. A61B 5/00; A61B 5/20; G16H 50/20

(52) U.S. Cl. CPC A61B 5/7267; A61B 5/202; A61B 5/7246; G16H 50/20

(57) ABSTRACT

A simple urine flow test result learning method and a lower urinary tract symptom diagnosis method is provided, and more particularly, a simple urine flow test result learning method and a lower urinary tract symptom diagnosis method of training a neural network using simple urine flow test results, which are non-invasive data, and diagnosing lower urinary tract symptoms using the trained neural network, wherein the lower urinary tract symptom diagnosis method prevents pain and shame from occurring in a patient during a diagnosis process of lower urinary tract symptoms and reduces the risk of secondary infection occurring through an invasive diagnosis method, by generating a trained model using results of a simple urine flow test, which is a non-invasive test method, based on deep learning, and diagnosing lower urinary tract symptoms using the trained model.

### FIELD OF THE INVENTION

The present invention relates to a simple urine flow test result learning method and a lower urinary tract symptom diagnosis method, and more particularly, to a simple urine flow test result learning method and a lower urinary tract symptom diagnosis method of training a neural network using simple urine flow test results, which are non-invasive data, and diagnosing lower urinary tract symptoms using the trained neural network.

### BACKGROUND OF THE INVENTION

Lower urinary tract symptoms (LUTS) refer to various symptoms related to the storage and excretion of urine, such as difficulty in starting, residual urine, frequent urination, thin urination, tension during urination, nocturia, urgency to urinate, and intermittent urination. Recently, the incidence and severity of lower urinary tract symptoms are increasing for various reasons such as psychological stress, smoking, drinking, weight gain, lack of rest, and lack of exercise due to an increase in animal fat intake and an increase in social complexity. When lower urinary tract symptoms become severe, activity is restricted, and patients are always in a state of anxiety and tension, causing great mental stress to the patient. Waking up during sleep to go to the bathroom or lack of sleep due to this, as well as physical fatigue, causes various physical problems.

Lower urinary tract symptoms are diagnosed using a urodynamic study (UDS) that evaluates bladder function. The urodynamic study is mainly performed to determine prostate surgery, and is performed to discriminate patients with bladder outlet obstruction (BOO), which is known to have a higher surgical effect, from patients with only detrusor under-activity (DUA), which has a lower surgical effect.

However, a conventional urodynamic study consists of inserting a tube for measuring pressure into the bladder and anus, measuring the pressure while slowly filling the bladder with saline, and then measuring the pressure of the bladder while urinating. In other words, the urodynamic study currently used to diagnose lower urinary tract symptoms not only causes discomfort and embarrassment to the patient, but also carries the risk of infection because the urodynamic study is performed while a catheter is inserted for a long time, and has a problem of causing pain and shame to the patient.

Accordingly, there is a continuous demand for the development of a lower urinary tract symptom diagnosis method capable of accurately determining the state of a urinary system without causing pain and shame to the patient.

### SUMMARY OF THE INVENTION

In accordance with the demand for the development of a lower urinary tract symptom diagnosis method described above, the present invention provides a lower urinary tract symptom diagnosis method, which prevents pain and shame from occurring in a patient during a diagnosis process of lower urinary tract symptoms and reduces the risk of secondary infection occurring through an invasive diagnosis method, by generating a trained model using results of a simple urine flow test, which is a non-invasive test method, based on deep learning, and diagnosing lower urinary tract symptoms using the trained model.

An embodiment of the present invention may provide a deep learning-based simple urine flow test result learning method for diagnosing lower urinary tract symptoms.

The method according to an embodiment of the present disclosure may include: extracting character data from a result sheet obtained through a simple urine flow test; and generating a character trained model using the character data as learning data to extract feature points having a correlation with a cause of lower urinary tract symptoms from the character data.

The character data according to an embodiment of the present disclosure may include at least one of a point (Qmax) having a maximum urine flow rate during urination, a voiding time, post-void residual (PVR), and a bladder filling volume (BFV).

The method according to an embodiment of the present disclosure may include: extracting graph data from a result sheet obtained through a simple urine flow test; and generating a graph trained model using the graph data as learning data to extract feature points having a correlation with a cause of lower urinary tract symptoms from the graph data.

The graph data according to an embodiment of the present disclosure may include a voided volume over time or a voided rate over time.

The extracting of graph data further include extracting a point where fluctuation of a graph starts in the graph data as a starting point where urine starts to come out; extracting a point where fluctuation of a graph ends in the graph data as an ending point where urine ends; and pre-processing of extracting a section from the starting point to the ending point and inputting the section to the graph trained model. An embodiment of the present invention may provide a deep learning-based lower urinary tract symptom diagnosis method.

The method according to an embodiment of the present disclosure may include: generating a character trained model using the method, and generating a graph trained model using the method; receiving a simple urine flow test result sheet to be diagnosed by a lower urinary tract symptom diagnosis system; extracting the character data and the graph data from the result sheet; extracting a plurality of feature points having a correlation with a cause of lower urinary tract symptoms from the character data and the graph data, respectively, and integrating the feature points; and diagnosing whether the result sheet corresponds to the lower urinary tract symptoms by analyzing a correlation between the feature points and the lower urinary tract symptoms by the character trained model and the graph trained model.

The character data according to an embodiment of the present disclosure may include at least one of a point (Qmax) having a maximum urine flow rate during urination, a voiding time, post-void residual (PVR), and a bladder filling volume (BFV).

The graph data according to an embodiment of the present disclosure may include at least one of a voided volume over time and a voided rate over time.

The extracting of the feature points according to an embodiment of the present disclosure may further include extracting a point where fluctuation of a graph starts in the graph data as a starting point where urine starts to come out; extracting a point where fluctuation of a graph ends in the graph data as an ending point where urine ends; and pre-processing of extracting a section from the starting point to the ending point and inputting the section to the graph trained model.

The diagnosing according to an embodiment of the present disclosure may further include combining a result of the diagnosing with symptoms corresponding to the lower urinary tract symptoms and expressing them in binary or quaternary.

An embodiment of the present invention may provide a non-transitory computer-readable recording medium having recorded thereon a program for executing the method.

According to an embodiment of the present invention, there is an effect of minimizing the physical/mental pain of a test subject and diagnosing lower urinary tract symptoms using only non-invasive test result data.

In addition, according to an embodiment of the present invention, there is an effect of reducing time and cost by omitting unnecessary tests such as urodynamic studies.

### BRIEF DESCRIPTION OF DRAWINGS

FIG. 1 is an example of a general simple urine flow test result sheet used for generating a trained module and diagnosing lower urinary tract symptoms according to the present invention.

FIG. 2 is a flowchart of a simple urine flow test result learning method using the character data 2 according to the present invention.

FIG. 3 is a flowchart of a simple urine flow test result learning method using the graph data 3 according to the present invention.

FIG. 4 is a flowchart of a graph extraction operation according to the present invention according to the present invention.

FIG. 5 is view of a process of extracting the graph data 3 in a learning method and diagnosis method according to the present invention.

FIG. 6 is a flowchart of a deep learning-based lower urinary tract symptom diagnosis method according to the present invention.

FIG. 7 is a schematic view of an embodiment of a process of integrating extracted feature points in a lower urinary tract symptom diagnosis method according to the present invention.

FIG. 8 is a block diagram of a lower urinary tract symptom diagnosis system according to the present invention.

### DETAILED DESCRIPTION OF THE INVENTION

Hereinafter, the terms used in the specification will be briefly described, and the configuration and operation of a preferred embodiment of the present invention will be described in detail as a detailed description for carrying out the present invention.

General and widely used terms have been employed herein, in consideration of functions provided in the present invention, and may vary according to an intention of one of ordinary skill in the art, a precedent, or emergence of new technologies. In addition, in certain cases, a term which is not commonly used can be selected. In such a case, the meaning of the term will be described in detail at the corresponding portion in the description of the present invention. Therefore, the terms used herein should be defined based on the meanings of the terms and the descriptions provided herein.

In addition, unless explicitly described to the contrary, the word “comprise” and variations such as “comprises” or “comprising” will be understood to imply the inclusion of stated elements but not the exclusion of any other elements. In addition, terms such as “ . . . unit”, “ . . . module”, or the like described herein refer to units that perform at least one function or operation, and the units may be implemented as hardware or software or as a combination of hardware and software. In addition, throughout the specification, when an element is “connected” to another element, the elements may not only be “directly connected”, but may also be “electrically connected” via another element therebetween.

Reference will now be made in detail to embodiments, examples of which are illustrated in the accompanying drawings. In this regard, the present invention may have different forms and should not be construed as being limited to the descriptions set forth herein. In addition, descriptions of well-known functions and constructions will be omitted for clarity and conciseness, and similar reference numerals are assigned to similar elements throughout the specification.

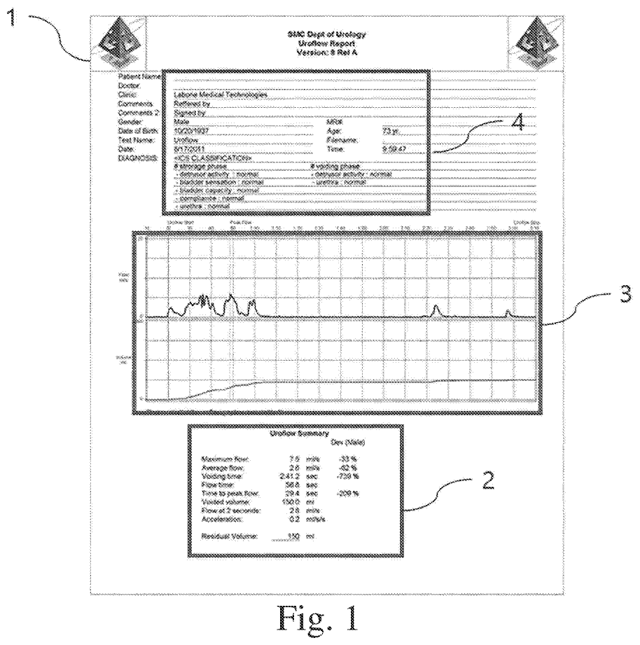

FIG. 1 is an example of a general simple urine flow test result sheet used for generating a trained module and diagnosing lower urinary tract symptoms.

Referring to FIG. 1, a simple urine flow test result sheet 1 used as input data for a simple urine flow test result learning method and a lower urinary tract symptom diagnosis method according to the present invention includes character data 2, graph data 3, and patient personal information 4.

The simple urine flow test result sheet 1 is a document in the form of reporting result data generated through a simple urine flow test.

The character data 2 includes at least one of a point (Qmax) having a maximum urine flow rate during urination, a voiding time, post-void residual (PVR), and a bladder filling volume (BFV).

The graph data 3 includes a voided volume over time or a voided rate over time.

The patient personal information 4 includes at least one of the age, height, and weight of a test subject, and a urination pattern, a urine flow test index, a prostate symptom score, past medical history, and voiding efficacy of the test subject obtained through a simple urine flow test.

In the character data 2, the point (Qmax) having a maximum urine flow rate during urination and the urination time may be used as elements to quantify the height and width of a graph in the graph data 3.

In addition, the bladder filling volume (BFV) is the sum of the voided volume and the post-void residual (PVR), so the degree of bladder fullness before voiding may be used as additional information on the assumption that the maximum urine flow rate and the pattern of urine flow may change depending on the state of bladder fullness.

In addition, because the post-void residual (PVR) (or voiding efficiency) is a value obtained by dividing the voided volume by the bladder filling volume (BFV), the post-void residual (PVR) may be used as additional information. Through this, the voiding efficiency may be expressed as a separate value that is not reflected in the graph.

The simple urine flow test result learning method according to the present invention extracts the character data 2 and the graph data 3 from the simple urine flow test result sheet 1 and generates a character trained model and a graph trained model that learn how each test data correlates with lower urinary tract symptoms, and the lower urinary tract symptom diagnosis method diagnoses whether there are lower urinary tract symptoms only with the simple urine flow test result sheet 1 by using a trained model generated by the simple urine flow test result learning method.

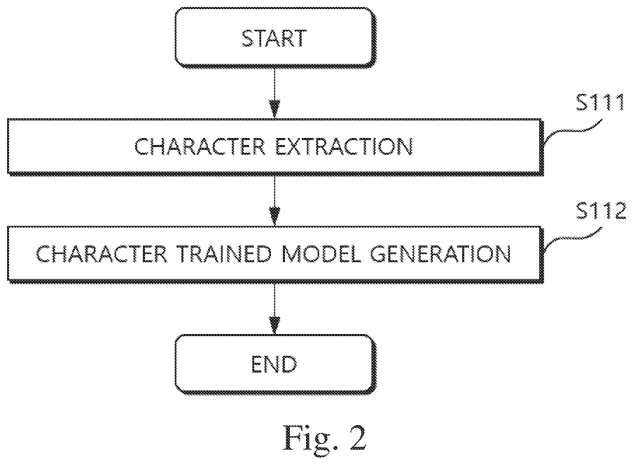

FIG. 2 is a flowchart of a simple urine flow test result learning method using the character data 2.

Referring to FIG. 2, the simple urine flow test result learning method using the character data 2 according to an embodiment of the present invention includes character extraction operation (S111) of extracting the character data 2 from a result sheet obtained through a simple urine flow test, and character trained model generation operation (S112) of generating a character trained model using the character data 2 as learning data to extract feature points having correlations according to causes of lower urinary tract symptoms from the character data 2.

In the character extraction operation (S111), the character data 2 including urination information of a patient, such as a point (Qmax) having a maximum urine flow rate during urination, a voiding time, post-void residual (PVR), and a bladder filling volume (BFV), is extracted from the simple urine flow test result sheet.

As a method of extracting the character data 2, a method of finding a portion containing the same string as the string of information to be extracted from the entire simple urine flow test result sheet and extracting urination information of the corresponding portion, a method of finding a location where the character data 2 is located in the simple urine flow test result sheet and extracting information corresponding to a certain range based on the location, or a function for finding the character data 2 may be used.

In the character trained model generation operation (S112), features of pieces of urination information of the input character data 2 are automatically extracted using deep learning.

That is, by looking at the pieces of urination information, it is possible to know the characteristics of the pieces of urination information that are meaningful for diagnosing lower urinary tract symptoms without a person directly comparing, analyzing, and determining criteria used for diagnosing lower urinary tract symptoms.

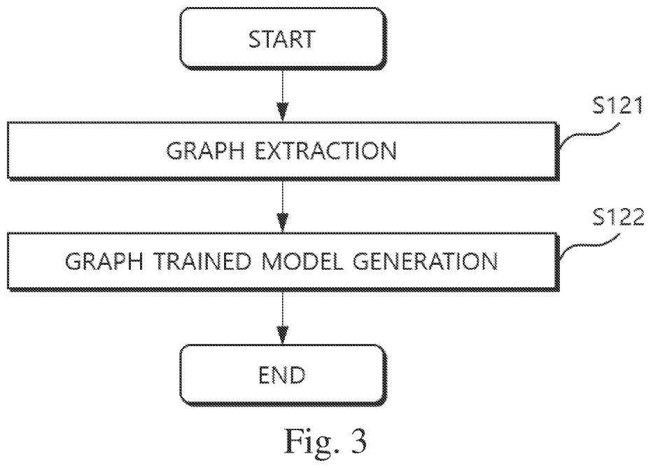

FIG. 3 is a flowchart of a simple urine flow test result learning method using the graph data 3.

Referring to FIG. 3, a deep learning-based simple urine flow test result learning method using the character data 2 according to an embodiment of the present invention includes graph extraction operation (S121) of extracting the graph data 3 from a result sheet obtained through a simple urine flow test, and graph trained model generation operation (S122) of generating a graph trained model using the graph data 3 as learning data to extract feature points having correlations according to causes of lower urinary tract symptoms from the graph data 2.

In the graph extraction operation (S121), the graph data 3 is extracted from a simple urine flow test result sheet. As a method of extracting the graph data 3, a method of receiving a location where the graph data 3 is located in the simple urine flow test result sheet and extracting information corresponding to a certain range based on the location, or a function to find the graph data 3 may be used.

In the graph trained model generation operation (S122), features of the input graph data 3 are automatically extracted using deep learning. A convolutional neural network (CNN), which is a neural network that borrows the principle of processing and recognizing images in the visual cortex of the brain, may be used to generate a graph trained model.

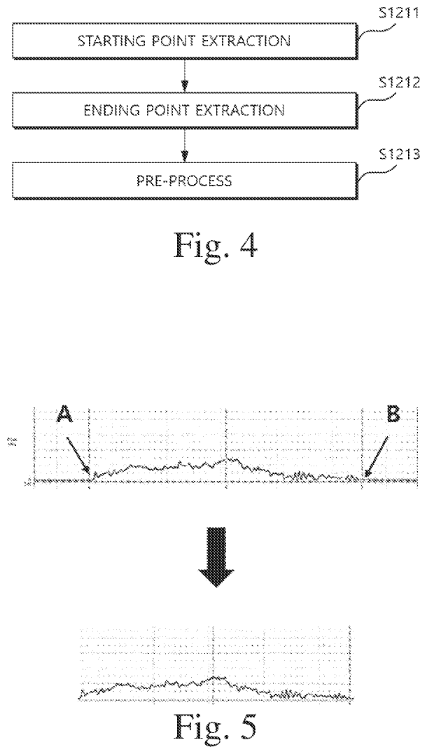

FIG. 4 is a flowchart of a graph extraction operation according to the present invention.

FIG. 5 is view of a process of extracting the graph data 3 in a learning method and diagnosis method according to the present invention.

Referring to FIG. 4, the graph extraction operation (S121) further includes starting point extraction operation (S1211) of extracting a point where the fluctuation of a graph starts in the graph data 3 as a starting point where urine starts to come out, ending point extraction operation (S1212) of extracting a point where the fluctuation of a graph ends in the graph data 3 as an ending point where urine ends, and pre-processing operation (S1213) of extracting a section from the starting point to the ending point and inputting the section to the graph trained model.

Referring to FIG. 5, in the graph data 3 extracted from a simple urine flow test result sheet, the amount of urine drained into a simple urine flow test device and the rate of urination from the start of the test to the end of the test are recorded. In general, the time at which the test starts and the time at which urination starts do not coincide, and the time at which urination ends and the time at which the test ends do not coincide. Accordingly, unnecessary information may be included in the graph data 3 between the time the test starts and the time urination starts, and between the time urination ends and the test ends.

In order to remove unnecessary information, in the present invention, a point where the fluctuation of a graph starts and a point where the fluctuation of a graph ends in the graph data 3 is defined as a starting point A and an ending point B, respectively, and a section between the starting point and the ending point is extracted and used to generate a trained model. Therefore, according to the present invention, more accurate information about the patient's urination volume and urination rate may be obtained.

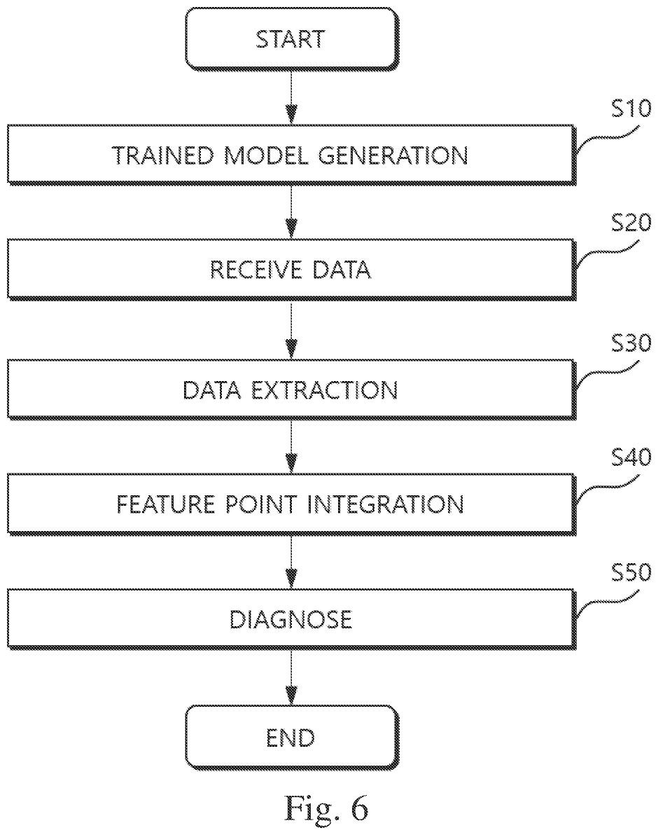

FIG. 6 is a flowchart of a deep learning-based lower urinary tract symptom diagnosis method according to the present invention.

Referring to FIG. 6, a simple urine flow test result learning method according to an embodiment of the present invention includes trained model generation operation (S10) of generating a character trained model and a graph trained model using a simple urine flow test result learning method according to an embodiment of the present invention, receiving operation (S20) of receiving a simple urine flow test result sheet to be diagnosed by a lower urinary tract symptom diagnosis system, data extraction operation (S30) of extracting the character data 2 and the graph data 3 from the result sheet, feature point integration operation (S40) of extracting a plurality of feature points having a correlation with the cause of lower urinary tract symptoms from the character data 2 and the graph data 3, respectively, and integrating the feature points, and diagnosing operation (S50) of diagnosing whether the result sheet corresponds to the lower urinary tract symptoms by analyzing a correlation between the feature points and the lower urinary tract symptoms by the character trained model and the graph trained model.

In the trained model generation operation (S10), the character trained model generated according to an embodiment of the present invention and the graph trained model generated according to an embodiment of the present invention are used together.

That is, the present invention may reduce errors that occur when only image data is used and errors that occur when only character data is used by using both character and image data instead of using only character data or image data.

Data received in the receiving operation (S20) is a simple urine flow test result sheet, and includes data measured only by the process of urinating into a toilet for examination connected to a computer recording device without the process of inserting a catheter into the patient's urethra and anus.

That is, the present invention may accurately diagnose lower urinary tract symptoms through deep learning while eliminating the possibility of pain and urinary tract infection, which are complications associated with catheter insertion. In addition, the present invention may reduce the time and cost required for diagnosing lower urinary tract symptoms by reducing unnecessary processes such as inserting a catheter into the patient's urethra and anus.

In the data extraction operation (S30), the character data 2 including urination information of the patient and the graph data 3 including information about the patient's urination volume and urination rate are extracted from the simple urination test result sheet.

The feature point integration operation (S40) is integrating feature points of character data and graph data generated by the character trained model and the graph trained model. This will be described in detail with reference to FIG. 7 below.

The diagnosing operation (S50) further includes combining a result of the diagnosing with symptoms corresponding to the lower urinary tract symptoms and expressing them in binary or quaternary. Examples of the symptoms corresponding to the lower urinary tract symptoms include bladder outlet obstruction (BOO) and detrusor underactivity (DUA).

That is, in the diagnosing operation (S50), a result of the diagnosing may be expressed in binary such as normal vs abnormal/BOO vs Non-BOO/DUA vs Non-DUA, or in quaternary such as normal vs BOO vs DUA vs BOO&DUA.

FIG. 7 is a view of an embodiment of a process of integrating extracted feature points in a lower urinary tract symptom diagnosis method according to the present invention.

Referring to FIG. 7, in a lower urinary tract symptom diagnosis method according to an embodiment of the present invention, in the feature point integration operation (S40), features extracted from the character data 2 may be integrated by applying a concatenate function to an intermediate layer of a graph trained model that takes the graph data 3 as an input or by performing element-wise addition.

In addition, each of the features extracted from the graph data 3 and the character data 2 may be integrated by applying a concatenate function right before an output layer of a character trained model or by performing element-wise addition.

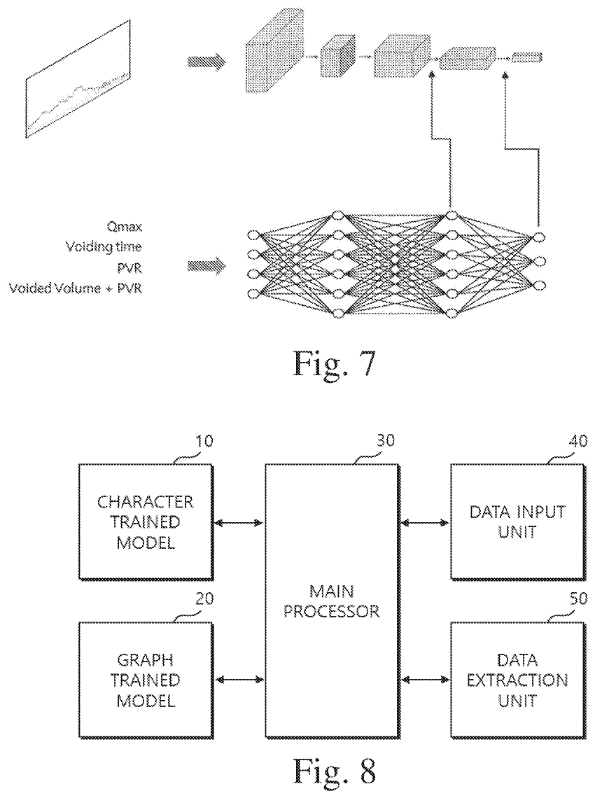

FIG. 8 is a block diagram of a lower urinary tract symptom diagnosis system according to the present invention.

Referring to FIG. 8, a lower urinary tract symptom diagnosis system according to an embodiment of the present invention includes a character trained model 10 that learns feature points by extracting the character data 2 from a simple urine flow test result sheet, a graph trained model 20 that learns feature points by extracting the graph data 3 from the result sheet, a data input unit 40 receiving the simple urine flow test result sheet, a data extraction unit 50 that extracts the character data 2 and the graph data 3 including patient's urination information from the simple urine flow test result sheet, and transmits the data to a trained model or main processor according to a learning or diagnosis process, and a main processor 30 that analyzes the simple urine flow test result sheet using the feature points learned from the trained model and determines whether there are lower urinary tract symptoms.

Regarding the lower urinary tract symptom diagnosis system according to the present invention, the method described above may be applied. Accordingly, in the description of the lower urinary tract symptom diagnosis system, descriptions of the same contents as those described above are omitted.

Although the present invention has been described in detail through representative embodiments above, one of ordinary skill in the art will understand that various modifications are possible without departing from the scope of the present invention in relation to the above-described embodiments. Therefore, the scope of the present invention should not be limited to the described embodiments, and should be defined by all changes or modifications derived from the claims and equivalent concepts as well as the claims to be described later.

1. A deep learning-based simple urine flow test result learning method for diagnosing lower urinary tract symptoms, the method comprising:
extracting character data from a result sheet obtained through a simple urine flow test; and
generating a character trained model using the character data as learning data to extract feature points having a correlation with a cause of lower urinary tract symptoms from the character data.

2. The method of claim 1, wherein the character data comprises at least one of a point (Qmax) having a maximum urine flow rate during urination, a voiding time, post-void residual (PVR), and a bladder filling volume (BFV).

3. A deep learning-based simple urine flow test result learning method for diagnosing lower urinary tract symptoms, the method comprising:
extracting graph data from a result sheet obtained through a simple urine flow test; and
generating a graph trained model using the graph data as learning data to extract feature points having a correlation with a cause of lower urinary tract symptoms from the graph data.

4. The method of claim 3, wherein the graph data comprises a voided volume over time or a voided rate over time.

5. The method of claim 3, wherein the extracting of graph data further comprises:
extracting a point where fluctuation of a graph starts in the graph data as a starting point where urine starts to come out;
extracting a point where fluctuation of a graph ends in the graph data as an ending point where urine ends; and
pre-processing of extracting a section from the starting point to the ending point and inputting the section to the graph trained model.

6. A deep learning-based lower urinary tract symptom diagnosis method comprising:
generating a character trained model using the method comprising:
extracting character data from a result sheet obtained through a simple urine flow test; and
generating a character trained model using the character data as learning data to extract feature points having a correlation with a cause of lower urinary tract symptoms from the character data;
generating a graph trained model using the method comprising:
extracting graph data from a result sheet obtained through a simple urine flow test; and
generating a graph trained model using the graph data as learning data to extract feature points having a correlation with a cause of lower urinary tract symptoms from the graph data;
receiving a simple urine flow test result sheet to be diagnosed by a lower urinary tract symptom diagnosis system;
extracting the character data and the graph data from the result sheet;
extracting a plurality of feature points having a correlation with a cause of lower urinary tract symptoms from the character data and the graph data, respectively, and integrating the feature points; and
diagnosing whether the result sheet corresponds to the lower urinary tract symptoms by analyzing a correlation between the feature points and the lower urinary tract symptoms by the character trained model and the graph trained model.

7. The method of claim 6, wherein the character data comprises at least one of a point (Qmax) having a maximum urine flow rate during urination, a voiding time, post-void residual (PVR), and a bladder filling volume (BFV).

8. The method of claim 6, wherein the graph data comprises at least one of a voided volume over time and a voided rate over time.

9. The method of claim 6, wherein the extracting of the feature points further comprises:
extracting a point where fluctuation of a graph starts in the graph data as a starting point where urine starts to come out;
extracting a point where fluctuation of a graph ends in the graph data as an ending point where urine ends; and
pre-processing of extracting a section from the starting point to the ending point and inputting the section to the graph trained model.

10. The method of claim 6, wherein the diagnosing further comprises:
combining a result of the diagnosing with symptoms corresponding to the lower urinary tract symptoms and expressing them in binary or quaternary.

11. A non-transitory computer-readable recording medium having recorded thereon a program for executing the method of claim 6.
